(19) United States
(12) Patent Application Publication
(10) Pub. No.: US 2022/0365057 A1
(43) Pub. Date: Nov. 17, 2022

(54) WATER QUALITY INSPECTION DEVICE

(71) Applicant: PiQuant. Co., Ltd., Seoul (KR)

(72) Inventor: Doyeon PI, Seoul (KR)

(73) Assignee: PiQuant. Co., Ltd., Seoul (KR)

(21) Appl. No.: 17/334,279

(22) Filed: May 28, 2021

(30) Foreign Application Priority Data
May 17, 2021 (KR) 10-2021-0063350

(51) Int. Cl. G01N 33/18; G01N 31/22

(52) U.S. Cl. CPC G01N 33/18; G01N 31/22

(57) ABSTRACT

The present invention relates to a water quality inspection device. The water quality inspection device, which inspects the water quality of drinking water by detecting whether a total coliform group is contained in the drinking water, includes a body portion in which an enzyme for detecting whether the total coliform group is accommodated, a suction part connected to the body portion and configured to suction the drinking water into the body portion, and a fixing part which is detachably attached to the body portion and fixes the body portion to the human body, wherein the water quality of the drinking water is inspected through a change in color of the drinking water due to the enzyme.

### CROSS-REFERENCE TO RELATED APPLICATION

This application claims priority to and the benefit of Korean Patent Application No. 10-2021-0063350, filed on May 17, 2021, the disclosure of which is incorporated herein by reference in its entirety.

### BACKGROUND

### 1. Field of the Invention

The present invention relates to a water quality inspection device.

### 2. Discussion of Related Art

According to a report of the World Health Organization (WHO), currently, more than one-third of the world's population do not have access to modern sanitation facilities, and 780 million people do not have access to adequate drinking water. The rate of children who drink contaminated drinking water and die due to waterborne diseases is one every 90 seconds, and 3.4 million people worldwide die due to the waterborne diseases. Particularly, this becomes a more serious problem for people mostly infected by the contaminated drinking water in developing countries. Diseases caused by drinking the contaminated drinking water include cholera, viral hepatitis, typhoid fever, and acute diarrhea, and among them, most diseases can be prevented with only awareness of the contaminated water and a facility or device for checking the contaminated water. That is, in order to prevent the waterborne disease, it is essential to check for fatal contamination.

However, test methods required to detect waterborne pathogenic microorganisms are time consuming and expensive. Accordingly, it is very important to check for contamination of drinking water by inspecting indicator microorganisms.

Escherichia coli (E. coli) kits for checking whether the E. coli group is contained in drinking water to check for contamination of drinking water are available on the market, and a consistent and suitable temperature should be maintained for the E. coli kit to consistently culture the E. coli. However, in facilities of developing countries where it is difficult to supply a constant and suitable temperature, there is a problem in that it is difficult to produce an accurate result even using the E. coli kit.

Accordingly, people in developing countries lacking resources urgently need a device that can be easily manufactured, distributed, and used to check whether water is contaminated.

### SUMMARY OF THE INVENTION

Therefore, the present invention is directed to providing a water quality inspection device which cultures total coliforms using a body temperature to check whether water is contaminated.

Objectives to be solved by the present invention are not limited to the above-described objectives, and other objectives, which are not described above, will be clearly understood by those skilled in the art through the following descriptions.

According to an aspect of the present invention, there is provided a water quality inspection device which inspects the water quality of drinking water by detecting whether a total coliform group is contained in the drinking water, the water quality inspection device including a body portion in which an enzyme for detecting whether the total coliform group is accommodated, a suction part connected to the body portion and configured to suction the drinking water into the body portion, and a fixing part which is detachably attached to the body portion and fixes the body portion to the human body, wherein water quality of the drinking water is inspected through a change in color of the drinking water due to the enzyme.

In a case in which the total coliform group is contained in the drinking water, the total coliform group may be cultured by the enzyme within a predetermined temperature range, and the color of the drinking water may be changed to a yellow color.

The suction part may be provided to be inserted into the body portion and to protrude outward from the body portion.

The fixing part may have a steel plate structure having a concave cross section, may be formed as a snap band type which is rolled when bent, may maintain a bent state when the fixing part having a linear shape in an unbent state is bent as a curved shape, and may maintain a linear state when the fixing part having the curved shape in the bent state is unbent as the linear shape.

The fixing part may include a first fixing member of which one end is connected to a first connecting member of the body portion and in which a first coupling member is formed on the other end, and a second fixing member of which one end is connected to a second connecting member of the body portion and in which a second coupling member is formed on the other end, wherein the first coupling member and the second coupling member may be provided to be connectable to each other.

The first connecting member may include a first coupling groove, the second connecting member may include a second coupling groove, the first fixing member may further include a first sliding portion provided to be detachably attached to the first coupling groove, and the second fixing member may further include a second sliding portion provided to be detachably attached to the second coupling groove.

The body portion may further include a crease portion on one surface thereof, and the crease portion may be spread out when the body portion is mounted on the human body.

The water quality inspection device may further include a cover part which is detachably connected to the body portion or the fixing part to cover one surface of the body portion.

The fixing part may further include a cover part of which one end is connected to the one end of the first fixing member and the other end is connected to the one end of the second fixing member, and the cover part may be formed to cover one surface of the body portion.

The water quality inspection device may further include a lamp configured to emit ultraviolet rays into the body portion, wherein the lamp may be provided on at least one of the body portion and the fixing part and may include a solar panel.

The body portion may further include a temperature sensor which measures a temperature of the drinking water inside the body portion, and a notification part which transmits a notification to a wearer when a measured temperature is beyond a predetermined range.

The water quality inspection device may further include a cap which opens or closes the suction part.

The other specific contents of the present invention will be included in the detailed description and the accompanying drawings.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a view illustrating a water quality inspection device according to an embodiment of the present invention;

FIG. 2A is a view illustrating a crease portion according to the embodiment of the present invention;

FIG. 2B is a view illustrating the crease portion according to the embodiment of the present invention;

FIG. 3 is a view illustrating a shape of a fixing part according to the embodiment of the present invention;

FIG. 4 is a view illustrating a body portion being connected to the fixing part according to the embodiment of the present invention;

FIG. 5 is a view illustrating a structure of a cover part according to the embodiment of the present invention;

FIG. 6 is a view illustrating a cap according to the embodiment of the present invention;

FIG. 7 is a view illustrating a shape of a suction part according to the embodiment of the present invention;

FIG. 8 is a view illustrating a lamp according to the embodiment of the present invention;

FIG. 9 is a view illustrating a temperature sensor and a notification part according to the embodiment of the present invention; and

FIG. 10 is a view illustrating a structure of the fixing part according to the embodiment of the present invention.

### DETAILED DESCRIPTION OF EXEMPLARY EMBODIMENTS

Advantages and features of the present invention and methods of achieving the same will be clearly understood with reference to the following embodiments and the accompanying drawings. However, the present invention is not limited to the embodiments to be disclosed below and may be implemented in various different forms, the embodiments are provided in order to fully describe the present invention and fully describe the scope of the present invention for those skilled in the art, and the scope of the present invention is only defined by the appended claims.

The terms used herein are provided only to describe the specific embodiments of the present invention and are not for purposes of limitation. Unless the context clearly indicates otherwise, the singular forms described in the specification include the plural forms. In addition, the terms “comprise” and/or “comprising” used herein do not preclude the presence or addition of one or more other components in addition to described components. Throughout the specification, the same reference symbol refers to the same component in the drawings, and the term “and/or” includes any and all combinations of the mentioned components. Although terms such as “first,” “second,” or the like may be used for describing various components, the components are not limited to the terms. The terms are only used to distinguish one component from another component. Accordingly, a first component described below may also be a second component within the technical spirit of the present invention.

Unless otherwise defined, all terms (including technical and scientific terms) used herein can be used with meanings that may be commonly understood by those of ordinary skill in the art to which this invention belongs. Also, terms defined in commonly used dictionaries will not be interpreted in an idealized or overly formal sense unless expressly defined herein.

Spatially relative terms such as “below,” “beneath,” “lower,” “above,” “upper,” and the like may be used to more easily describe a relationship between one component and other components as illustrated in the drawings. The spatially relative terms should be understood to have directions as illustrated in the drawings and have other directions when the components are used or operated. For example, when an upper side of a component illustrated in the drawing is inverted to face downward, the component which is illustrated as being present below or beneath another component may be present above the other component. Accordingly, the term “below” may be used as an example including both a downward direction and an upward direction. A component may be oriented in another direction, and thus, the spatially relative terms may be interpreted based on an orientation.

Hereinafter, an embodiment of the present invention will be described in detail with reference to the accompanying drawings.

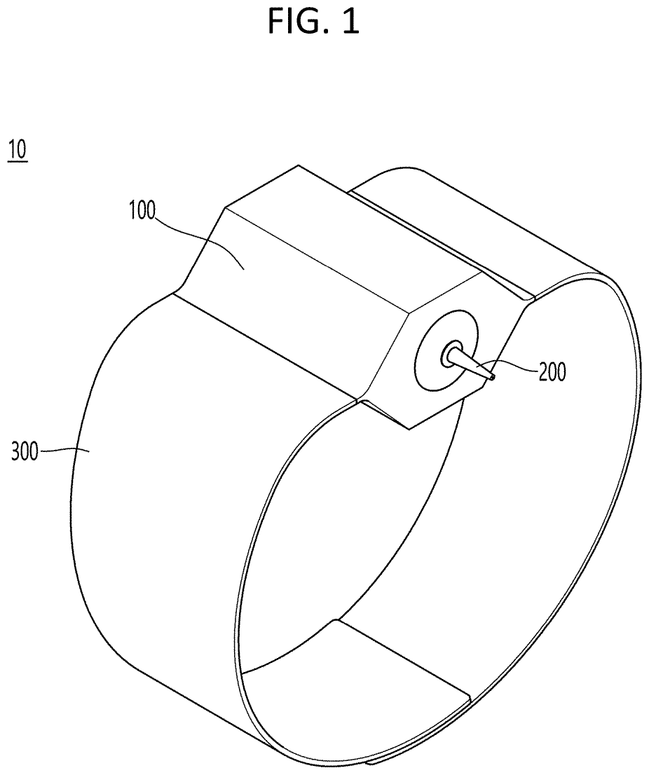

FIG. 1 is a view illustrating a water quality inspection device according to an embodiment of the present invention.

Referring to FIG. 1, a water quality inspection device 10 according to the embodiment of the present invention may include a body portion 100, a suction part 200, and a fixing part 300.

The body portion 100 may accommodate an enzyme for detecting whether the total coliform group is contained. The body portion 100 may have a tub shape in which the enzyme and drinking water are accommodated. That is, the body portion 100 may have an empty interior so that it is possible to accommodate the enzyme and drinking water and may be formed of a transparent material so that a state of the enzyme accommodated therein is checkable. That is, in a case in which the total coliform group is contained in the drinking water, the total coliform group is cultured by the enzyme, and thus, the total coliform group may become yellow in color. The body portion 100 may be formed of the transparent material so that such a state may be immediately and visually checked.

The total coliform group may be cultured by the enzyme in a predetermined temperature range, and the predetermined temperature range may be similar to a body temperature. Accordingly, the water quality inspection device 10 according to the embodiment of the present invention may be implemented to be fixed while in contact with the human body so that a body temperature is transferred into the body portion 100.

The fixing part 300 may be connected to two corners among corners of side surfaces of the body portion 100. In this case, two other corners may be present between the two corners connected to the fixing part 300. A surface including the two other corners between the two corners connected to the fixing part 300 may be a surface in contact with the skin of a wearer. That is, the body portion 100 may have a concave shape when viewed from a viewpoint of the wearer, and the wearer may wear the water quality inspection device 10 to be in contact with at least a part of the body portion 100.

Meanwhile, the fixing part 300 may be connected to two consecutive corners among the corners of the side surfaces of the body portion 100. In this case, the surface including the two corners connected to the fixing part 300 may be a surface in contact with the skin of the wearer.

The body portion 100 is described as having a hexagonal column shape, but the shape of the body portion 100 may not be limited thereto. For example, the body portion 100 may have a trapezoidal column, quadrangular column, or elliptical sphere shape.

In addition, the body portion 100 may be formed to have a curvature to be worn on and pressed against the human body, and thus more areas can be in contact with the human body to more easily maintain the predetermined temperature range. The suction part 200 may be connected to the body portion 100 to suction drinking water into the body portion 100. The suction part 200 may suction the drinking water into the body portion 100, and the drinking water may be mixed with an enzyme accommodated in the body portion 100. Accordingly, in a case in which the total coliform group is contained in the drinking water, the total coliform group may be cultured by the enzyme and become yellow in color.

According to the present invention, the suction part 200 may be formed to protrude from the body portion 100. The suction part 200 may be formed in a pipette shape, and accordingly, in a state in which the body portion 100 is pressed, the suction part 200 may be brought into contact with the drinking water, and the pressure applied to the body portion 100 may be gradually reduced so that the drinking water may be suctioned through the suction part 200 and introduced into the body portion 100. An inlet of the suction part 200 may be blocked, and when the water quality inspection device 10 is used, the inlet of the suction part 200 may be opened to suction drinking water. Accordingly, an enzyme accommodated in the body portion 100 may be prevented from being discharged through the suction part 200, or the enzyme may be prevented from being exposed to air and a state thereof is prevented from being changed before the water quality inspection device 10 is used. In this case, a component, which blocks the inlet of the suction part 200, may be formed of a disposable material, such as paper or plastic, which is detached and discarded. In addition, the component which blocks the inlet of the suction part 200 may denote a cap which will be described below.

The fixing part 300 may be detachably attached to the body portion 100 and may fix the body portion 100 to the human body. According to the embodiment of the present invention, in order to culture the total coliform group, a temperature of 35° C. to 36° C. should be maintained for 12 hours to 24 hours. Since the temperature of 35° C. to 36° C. is similar to a body temperature, the temperature of 35° C. to 36° C. may be maintained even when the water quality inspection device 10 is attached to the body portion 100. To this end, the fixing part 300 may fix the body portion 100 to the human body.

The fixing part 300 may be formed to be detachably attached to the body portion 100 in a sliding manner. That is, coupling grooves allowing the fixing part 300 to be coupled thereto may be formed in both side surfaces of the body portion 100, and the fixing part 300 may include sliding portions coupled to the coupling grooves of the body portion 100. The fixing part 300 and the body portion 100 may be detachably attached to each other by the coupling grooves and the sliding portions. However, the present invention is not limited thereto, and the fixing part 300 and the body portion 100 may be connected to each other by Velcro, a buckle, a button, or the like or may also be connected to each other by an adhesive.

According to the embodiment of the present invention, the fixing part 300 may be connected to be wrapped around an arm or leg of the wearer to fix the water quality inspection device 10 to the human body. The fixing part 300 may be formed with a snap band, when the wearer brings the fixing part 300 into contact with a portion on which the water quality inspection device 10 is worn, the fixing part 300 is rolled in a circular shape to be wrapped around the body of the wearer, and the water quality inspection device 10 may be fixed to the human body. Since the fixing part 300 is formed with the snap band, the wearer may simply fix the water quality inspection device 10 to the human body.

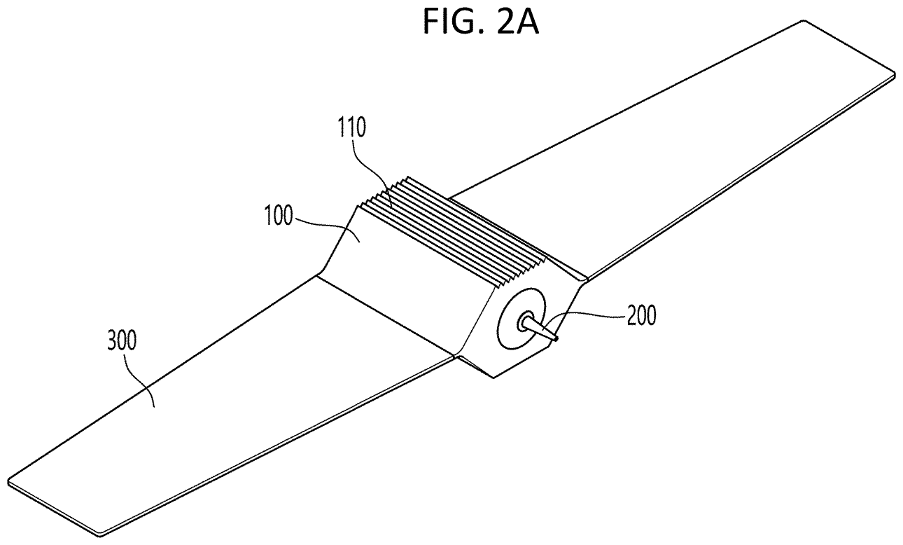

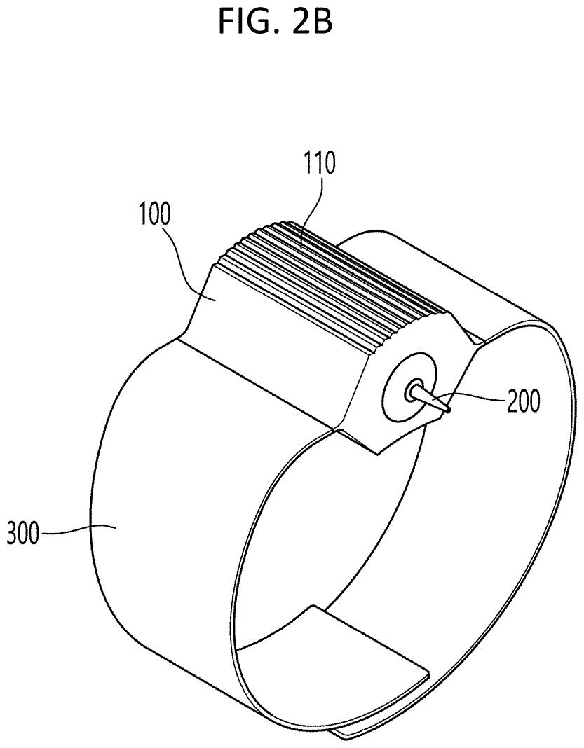

FIGS. 2A and 2B are views illustrating a crease portion according to the embodiment of the present invention.

FIG. 2A is a view illustrating a state in which the crease portion is not spread out, and FIG. 2B is a view illustrating a state in which the crease portion is spread out. Referring to FIGS. 2A and 2B, a crease portion 110 may be formed on one surface of the body portion 100 according to the embodiment of the present invention. In this case, one surface of the body portion 100 may be a surface which is not in contact with the human body. For example, the water quality inspection device 10 may be worn to be wrapped around the human body, and the wearer should wear the water quality inspection device 10 for 12 hours to 24 hours. Accordingly, it is important that the wearer does not feel inconvenience even when wearing the water quality inspection device 10 for a long time.

The crease portion 110 formed on the body portion 100 may be creased or spread out. For example, the crease portion 110 may be in a creased state before the water quality inspection device 10 is worn, and creases of the crease portion 110 may be spread out when the water quality inspection device 10 is worn. Accordingly, the body portion 100 may be in easy contact with the human body.

Meanwhile, it is described that the crease portion 110 is formed on one surface of the body portion 100, but the crease portion 110 is not limited thereto and may also be formed on the other surface opposite to the one surface. That is, the crease portion 110 may be formed on both surfaces of the body portion 100 and creased before the water quality inspection device 10 is worn, and since the body portion 100 is creased, a size of the water quality inspection device 10 may be decreased so that it may be easy to store and move. Meanwhile, in the case in which the water quality inspection device 10 is worn, when the crease portion 110 is spread out and the water quality inspection device 10 is attached to the human body, an increased area of the water quality inspection device 10 may be in contact with the human body. Accordingly, it may be more suitable to maintain a proper temperature.

In addition, as the creases of the crease portion 110 are contracted or expanded, an area ratio of the body portion 100 is changed, and thus, an amount of drinking water contained in the body portion 100 may also be changed.

In addition, when the creases of the crease portion 110 are spread out, a curvature may be formed on the body portion 100, and thus, a curvature may also be formed on the other surface of the body portion 100 like the crease portion 110. Since the curvature is formed on the other surface of the body portion 100, the body portion 100 may be more easily attached to the wearer's body.

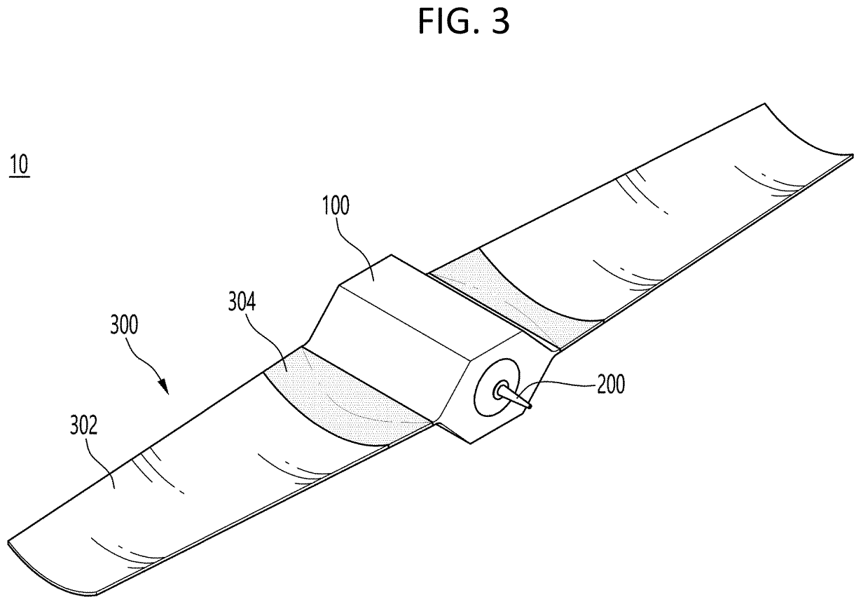

FIG. 3 is a view illustrating a shape of the fixing part according to the embodiment of the present invention.

Referring to FIG. 3, the fixing part 300 may include band parts 302 and connector parts 304. The band part 302 may be formed with an iron plate structure having a concave cross section and formed in a type which is rolled when bent. That is, when the band part 302 in a linearly unbent state is bent in a curved shape, the band part 302 may be maintained in a bent state. Meanwhile, when the band part 302 in the curved bent state is unbent in a linear shape, the band part 302 may be maintained in a linear state of.

According to the present invention, when the band part 302 is unbent in the linear shape, the cross section of the band part 302 may have a concave shape, and when the band part 302 is bent in the curved shape, the cross section of the band part 302 may have a flat shape. The fixing part 300 is a part connected to the body portion 100, but it may be difficult for the fixing part 300 to be connected to the body portion 100 due to the shape thereof being changed as described above.

Accordingly, the connector part 304, which is a part connected to the body portion 100, may be formed of a material, such as rubber, silicone, or cloth, of which a shape is easily changed. For example, one end of connector part 304 is connected to the band part 302, and the other end of the connector part 304 may be connected to the body portion 100. Accordingly, as the shape of the band part 302 is changed to the linear or curved shape, one end of the connector part 304 may also be changed according to the change in the shape of the band part 302. However, a state in which the other end of the connector part 304 is connected to the body portion 100 may be maintained. Accordingly, the body portion 100 and the fixing part 300 are connectable.

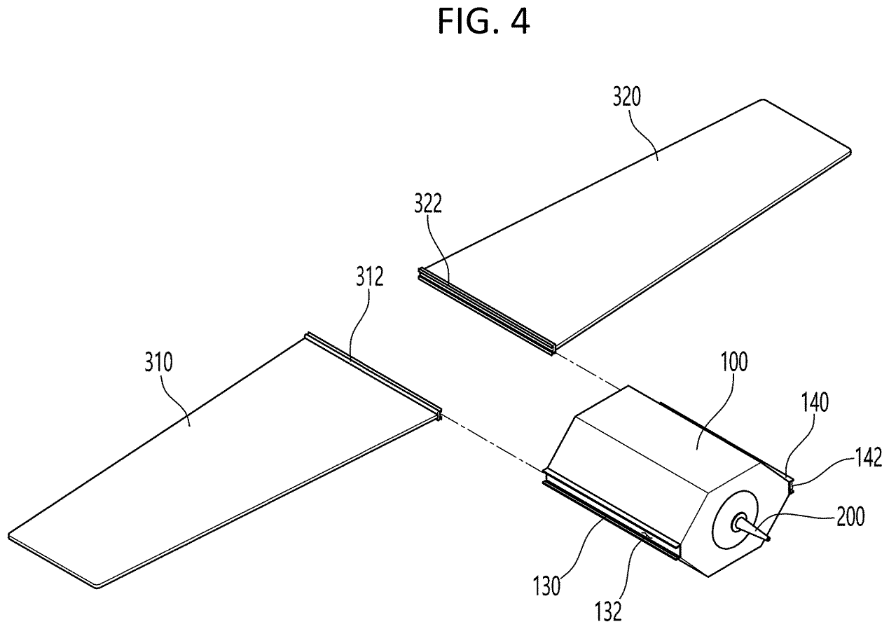

FIG. 4 is a view illustrating the body portion being connected to the fixing part according to the embodiment of the present invention.

Referring to FIG. 4, the fixing part 300 may include a first fixing member 310 and a second fixing member 320.

One end of the first fixing member 310 may be connected to a first connecting member 130 of the body portion 100. The first fixing member 310 and the body portion 100 may be detachably connected to each other. That is, a first coupling groove 132 may be formed in the first connecting member 130, and a first sliding portion 312 may be formed at one end of the first fixing member 310. As the first sliding portion 312 moves along the first coupling groove 132, the fixing part 300 may be connected to the body portion 100.

In addition, one end of the second fixing member 320 may be connected to a second connecting member 140 of the body portion 100. The second fixing member 320 and the body portion 100 may be detachably connected to each other. That is, a second coupling groove 142 may be formed in the second connecting member 140, and a second sliding portion 322 may be formed at one end of the second fixing member 320. As the second sliding portion 322 moves along the second coupling groove 142, the fixing part 300 may be connected to the body portion 100.

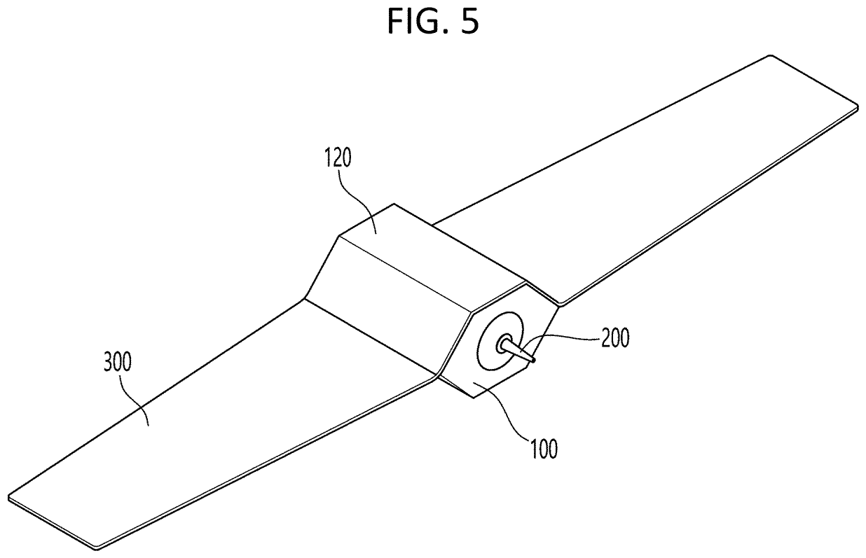

FIG. 5 is a view illustrating a structure of a cover part according to the embodiment of the present invention.

Referring to FIG. 5, a cover part 120 according to the embodiment of the present invention may be formed to cover one surface of the body portion 100. In order to culture the total coliform group contained in drinking water, it is important to maintain a predetermined temperature. Meanwhile, since a state in which the water quality inspection device 10 is attached to the human body should be maintained for a long time, it is easy to be affected by an external environment. For example, in the case of a day with strong sunlight, a temperature in the body portion 100 may be increased due to the influence of the sunlight. Meanwhile, in the case of a day with strong wind, a temperature in the body portion 100 may be decreased due to the influence of the wind. In order to minimize a change in temperature due to such external factors, the cover part 120 may be connected to the body portion 100.

The cover part 120 may be detachably connected to the body portion 100 or the fixing part 300. For example, one end of the cover part 120 may be connected to the first connecting member 130, and the other end of the cover part 120 may be connected to the second connecting member 140. In addition, one end of the cover part 120 may be connected to the first fixing member 310, and the other end of the cover part 120 may be connected to the second fixing member 320.

In addition, when one end of the cover part 120 is connected to the first fixing member 310, the other end of the cover part 120 is connected to the second fixing member 320, and the fixing part 300 is connected to the body portion 100, the cover part 120 may also be connected to the body portion 100. In this case, the cover part 120 may be integrally formed with the fixing part 300. In addition, in a state in which the fixing part 300 is connected to the body portion 100, the cover part 120 may also be individually connected to the body portion 100.

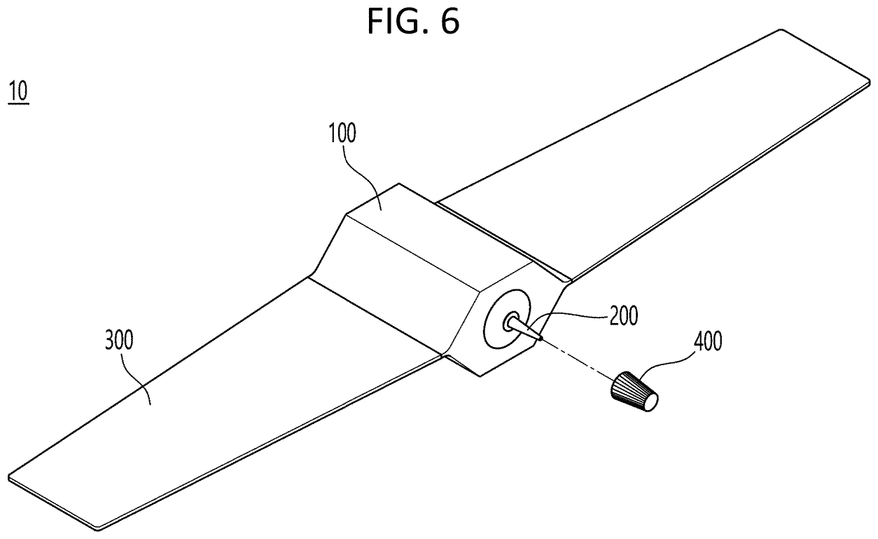

FIG. 6 is a view illustrating the cap according to the embodiment of the present invention.

Referring to FIG. 6, a cap 400 may block the inlet of the suction part 200. When drinking water is suctioned into the body portion 100 through the suction part 200, a mixed state of an enzyme and the drinking water should be maintained for a long time. That is, in order to culture the total coliform group which may be present in the drinking water, the enzyme and the drinking water contained in the body portion 100 should be prevented from being discharged through the suction part 200, and other bacteria should be prevented from being introduced into the body portion 100 to prevent an error in inspection results.

Accordingly, after the drinking water is suctioned, the inlet of the suction part 200 may be blocked using the cap 400. The cap 400 may be coupled to the suction part 200 when rotated in one direction or also coupled to the suction part 200 when pressed by a force.

After the cap 400 blocks the inlet of the suction part 200, the cap 400 may be separated from the suction part 200 only when a force is applied to the cap 400 or the cap 400 is rotated in a direction opposite to the one direction so that the content in the body portion 100 may not be discharged during a time in which the total coliform group is cultured.

Meanwhile, the cap 400 may be already coupled to the suction part 200 before the drinking water is put thereinto. That is, the cap 400 may be coupled to the suction part 200 in order to prevent an enzyme from being discharged outward when only the enzyme is contained in the body portion 100. The wearer may separate the cap 400, input drinking water into the body portion 100 through the suction part 200, and couple the cap 400 to the suction part 200 again.

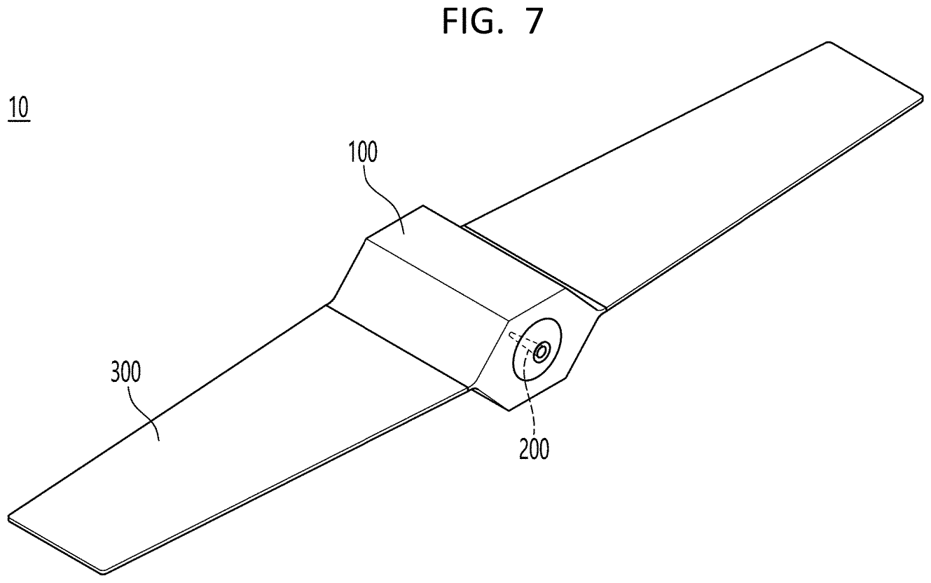

FIG. 7 is a view illustrating a shape of the suction part according to the embodiment of the present invention.

Referring to FIG. 7, the suction part 200 according to the embodiment of the present invention may be provided to be inserted into the body portion 100 and to protrude outward from the body portion 100. For example, the suction part 200 may have elasticity, and the wearer may push the suction part 200 into the body portion 100 after drinking water is suctioned through the suction part 200. Accordingly, the suction part 200 may be inserted into the body portion 100, and thus, even when the water quality inspection device 10 is fixed to the wearer's body, inconvenience due to the protruding portion can be reduced.

For example, when the suction part 200 is in a protruding state, a situation may occur in which the wearer is stuck by the suction part 200 when the human body moves, and in addition, a situation may also occur in which the suction part 200 is pressed to be bent or broken by the human body. Then, such a problem may be prevented by inserting the suction part 200 into the body portion.

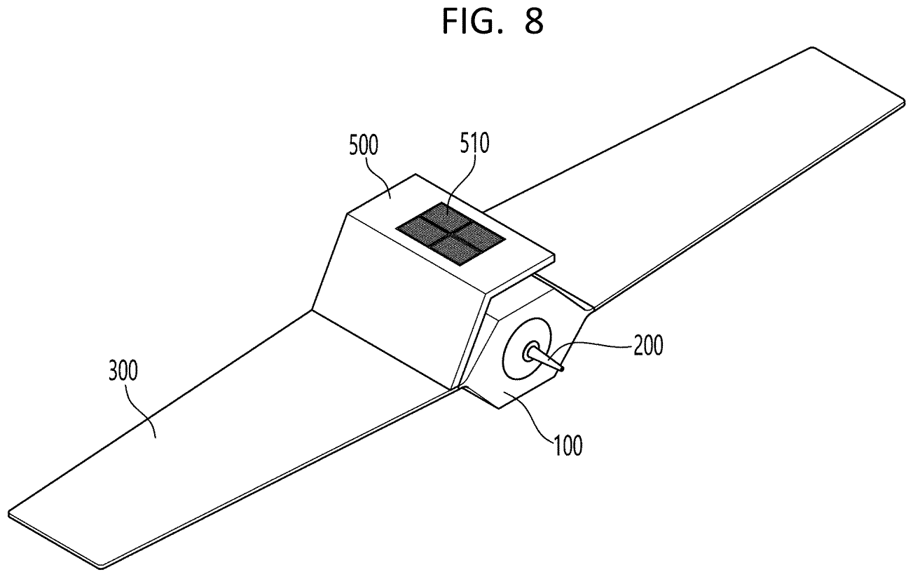

FIG. 8 is a view illustrating a lamp according to the embodiment of the present invention.

Referring to FIG. 8, the water quality inspection device 10 according to the embodiment of the present invention may include a lamp 500. The lamp 500 may be detachably attached to at least one of the body portion 100 and the fixing part 300. The lamp 500 may emit ultraviolet rays into the body portion 100. The lamp 500 may be a lamp for determining whether there is Escherichia coli (E. coli) among the total coliform group. The lamp 500 may emit light with a wavelength of 365 nm and, in a case in which drinking water contains E. coli, blue fluorescent light may be emitted due to the ultraviolet rays emitted from the lamp. Accordingly, the presence of E. coli can be determined.

Meanwhile, in order for the lamp 500 to emit the ultraviolet rays, power may be needed. However, in the case of a developing country lacking resources, power may not be received using electricity. Accordingly, a separate battery may be inserted into the lamp 500, and the ultraviolet rays may be emitted using the inserted battery. In addition, a solar panel 510 may also be installed on the lamp 500. Sunlight may be collected through the solar panel 510, and the collected sunlight may be converted into energy to supply power to the lamp 500.

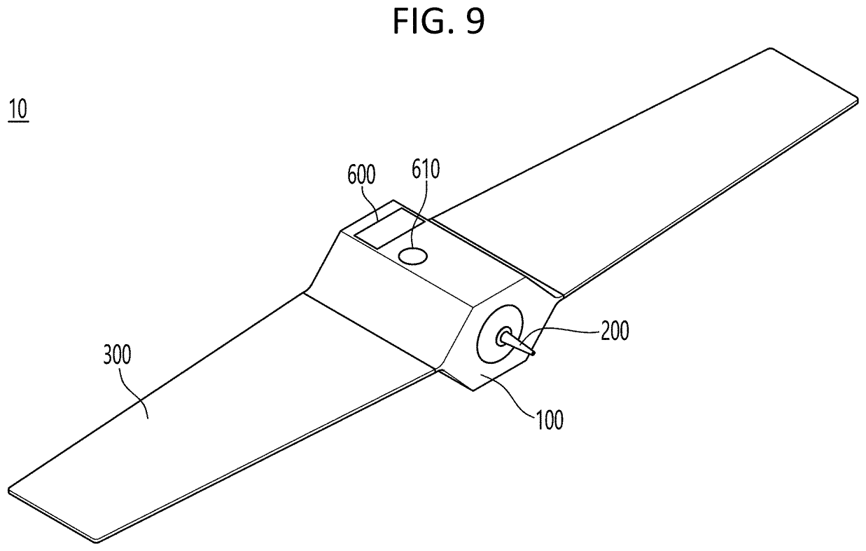

FIG. 9 is a view illustrating a temperature sensor and a notification part according to the embodiment of the present invention.

Referring to FIG. 9, the water quality inspection device 10 should maintain a temperature of 35° C. to 36° C. to culture the total coliform group contained in drinking water, and in order to maintain the temperature of 35° C. to 36° C., the water quality inspection device 10 may be in contact with the human body. However, even when the water quality inspection device 10 is in contact with the human body of which a temperature is similar to the temperature of 35° C. to 36° C., the temperature of the human body may be changed due to an external factor and the like, and the external factor may directly affect the water quality inspection device 10 to change the temperature thereof.

For example, in a case in which heat is generated from the wearer's body when the wearer enters cold or hot water or has a disease in a state in which the wearer wears the water quality inspection device 10, a body temperature may be raised or lowered. In this case, since a temperature for culturing the total coliform group is not satisfied, the water quality inspection device 10 should be worn for a longer time, and thus, a result of whether the total coliform group is contained may not come out normally.

Accordingly, the water quality inspection device 10 according to the embodiment of the present invention may include a temperature sensor 600 and a notification part 610.

The temperature sensor 600 may be attached to the body portion 100 and may measure a temperature of drinking water in the body portion 100.

In addition, in a case in which a temperature measured by the temperature sensor 600 is beyond a preset range, the notification part 610 may transmit an alarm to the wearer. For example, the notification part 610 may include a light-emitting diode (LED), a speaker, and the like, and in the case in which the temperature is beyond the preset range, the LED may be turned on to notify that the temperature is beyond the preset range, and a sound may be generated through the speaker to notify that the temperature is beyond the preset range.

Accordingly, even when the wearer does not frequently check a state of the water quality inspection device 10, the wearer may immediately recognize a case in which the temperature of the drinking water in the body portion is beyond the preset range, and by checking the temperature sensor 600, it possible to take an action so as to bring the temperature of the water quality inspection device 10 to within the preset range.

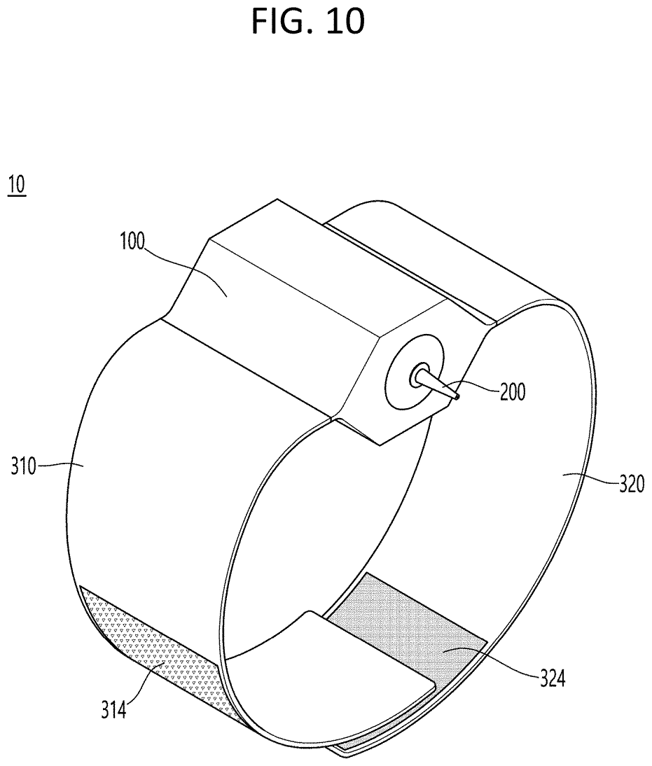

FIG. 10 is a view illustrating a structure of the fixing part according to the embodiment of the present invention.

Referring to FIG. 10, a first coupling member 314 may be formed at the other end of the first fixing member 310. In addition, a second coupling member 324 may be formed at the other end of the second fixing member 320.

The first coupling member 314 and the second coupling member 324 are connectable to each other, and thus, when the first coupling member 314 and the second coupling member 324 are connected to each other, the water quality inspection device 10 may be fixed to the human body.

The first coupling member 314 and the second coupling member 324 according to the embodiment of the present invention may each be formed as a Velcro type. In this case, Velcro may be formed on each of different surfaces of the first fixing member 310 and the second fixing member 320. For example, when Velcro is formed on one surface of the first fixing member 310, Velcro may be formed on the other surface of the second fixing member 320. That is, in a case in which a surface, on which Velcro is formed, of the first fixing member 310 is a surface which comes into contact with the human body, a surface, on which Velcro is formed, of the second fixing member 320 may be an externally exposed surface. In addition, in a case in which a surface, on which Velcro is formed, of the first fixing member 310 is an externally exposed surface, a surface, on which Velcro is formed, of the second fixing member 320 may be a surface which comes into contact with the human body.

Accordingly, when the first fixing member 310 and the second fixing member 320 are connected by Velcro, the fixing part 300 may be fixed to the human body. The wearer may adjust a size of the water quality inspection device 10 to be in exact contact with the wearer's body using the Velcro.

Meanwhile, it is described that the first coupling member 314 and the second coupling member 324 are each formed as the Velcro type, but the first coupling member 314 and the second coupling member 324 are not limited thereto.

For example, the first coupling member 314 may be a female button, and the second coupling member 324 may be a male button. In this case, in order to wear the water quality inspection device 10 to fit the wearer's body, a plurality of female buttons may be installed to be spaced apart from each other at predetermined intervals. The wearer may couple the male button to one of the plurality of female buttons to fix the fixing part 300 to the human body.

In addition, a coupling hole may be formed in the first coupling member 314, and the second coupling member 324 may include a coupling rod and a coupling ring. In this case, in order to wear the water quality inspection device 10 to fit the wearer's body, the coupling hole may be formed as a plurality of coupling holes. The wearer may bring the first fixing member 310 to pass through the coupling hole and insert the coupling rod into one coupling hole among the plurality of coupling holes so as to fix the fixing part 300 to the human body.

According to the present invention, only by putting drinking water in a device containing an enzyme, attaching the device on the human body, and maintaining for a predetermined time, the total coliform group can be detected so that whether water is contaminated can be determined.

In addition, since the drinking water is put into the device and the device is attached to the human body, water contamination can be inspected through only a simple action.

In addition, since the drinking water is put into the device and the device is attached to the human body, external energy such as electricity does not need to be used, and thus, the device can be used in developing countries lacking resources.

Effects of the present invention are not limited to the above-described effects, and other effects, which are not described, will be clearly understood by those skilled in the art from the above-described detailed description.

Although the embodiment of the present invention has been described with reference to the accompanying drawings as described above, it will be understood by those skilled in the art that the invention may be implemented in other concrete forms without changing the technological scope and essential features. Therefore, the above-described embodiments should be considered in an illustrative sense only in all respects and not for purposes of limitation.

What is claimed is:

1. A water quality inspection device which inspects water quality of drinking water by detecting whether a total coliform group is contained in the drinking water, the water quality inspection device comprising:
a body portion in which an enzyme for detecting whether the total coliform group is accommodated;
a suction part connected to the body portion and configured to suction the drinking water into the body portion; and
a fixing part which is detachably attached to the body portion and fixes the body portion to a human body,
wherein water quality of the drinking water is inspected through a change in color of the drinking water due to the enzyme.

2. The water quality inspection device of claim 1, wherein, in a case in which the total coliform group is contained in the drinking water,
the total coliform group is cultured by the enzyme within a predetermined temperature range, and
the color of the drinking water is changed to a yellow color.

3. The water quality inspection device of claim 1, wherein the suction part is provided to be inserted into the body portion and to protrude outward from the body portion.

4. The water quality inspection device of claim 1, wherein the fixing part has a steel plate structure having a concave cross section, is formed as a snap band type which is rolled when bent, maintains a bent state when the fixing part having a linear shape in an unbent state is bent as a curved shape, and maintains a linear state when the fixing part having the curved shape in the bent state is unbent as the linear shape.

5. The water quality inspection device of claim 1, wherein the fixing part includes:
a first fixing member of which one end is connected to a first connecting member of the body portion and in which a first coupling member is formed on the other end; and
a second fixing member of which one end is connected to a second connecting member of the body portion and in which a second coupling member is formed on the other end,
wherein the first coupling member and the second coupling member are provided to be connectable to each other.

6. The water quality inspection device of claim 5, wherein:
the first connecting member includes a first coupling groove;
the second connecting member includes a second coupling groove;
the first fixing member further includes a first sliding portion provided to be detachably attached to the first coupling groove; and
the second fixing member further includes a second sliding portion provided to be detachably attached to the second coupling groove.

7. The water quality inspection device of claim 1, wherein:
the body portion further includes a crease portion on one surface thereof; and
the crease portion is spread out when the body portion is mounted on the human body.

8. The water quality inspection device of claim 1, further comprising a cover part which is detachably connected to the body portion or the fixing part to cover one surface of the body portion.

9. The water quality inspection device of claim 5, wherein:
the fixing part further includes a cover part of which one end is connected to the one end of the first fixing member and the other end is connected to the one end of the second fixing member; and
the cover part is formed to cover one surface of the body portion.

10. The water quality inspection device of claim 1, further comprising a lamp configured to emit ultraviolet rays into the body portion,
wherein the lamp is provided on at least one of the body portion and the fixing part and includes a solar panel.

11. The water quality inspection device of claim 1, wherein the body portion further includes:
a temperature sensor which measures a temperature of the drinking water inside the body portion; and
a notification part which transmits a notification to a wearer when a measured temperature is beyond a predetermined range.

12. The water quality inspection device of claim 1, further comprising a cap which opens or closes the suction part.
